(19) United States
(12) Patent Application Publication
(10) Pub. No.: US 2015/0025563 A1
(43) Pub. Date: Jan. 22, 2015

(54) SYSTEMS AND METHODS FOR A TISSUE EXPANDER

(71) Applicant: Reconstructive Technologies, LLC, Phoenix, AZ (US)

(72) Inventors: Tamir M. Mosharrafa, Paradise Valley, AZ (US); Ali M. Mosharrafa, Paradise Valley, AZ (US)

(73) Assignee: Reconstructive Technologies, LLC, Phoenix, AZ (US)

(21) Appl. No.: 13/948,067

(22) Filed: Jul. 22, 2013

(51) Int. Cl. A61B 19/00

(52) U.S. Cl. CPC A61B 19/24
USPC 606/191

(57) ABSTRACT

Various embodiments provide systems and methods for a temporary extended tissue expander. Systems and methods according to various aspects of the present invention may comprise an extended tissue expander configured to increase and/or customize the size and/or shape of a tissue pocket beyond the size and/or shape provided by a conventional tissue expander. The extended tissue expander may comprise a tissue expander coupled to an extension portion. In one embodiment, the extended tissue expander may preserve a breast pocket with a desired teardrop shape that does not need extensive surgical modification before placement of a final breast implant or a tissue flap. In another embodiment, the extended tissue expander may be modified to the precise dimensions of a final breast implant for the simple exchange of the extended tissue expander for the final breast implant.

### CROSS-REFERENCE TO RELATED APPLICATION

This application claims the benefit of U.S. Provisional Application No. 61/810,160, filed Apr. 9, 2013, and incorporates the disclosure of such application in its entirety by reference. To the extent that the present disclosure conflicts with the referenced application, however, the present disclosure is to be given priority.

### BACKGROUND

Soft tissue expansion techniques are used in reconstructive surgeries to stimulate the growth of new skin to create a stretched area of skin for placement of a prosthetic implant. A tissue expander device comprising an expandable balloon may be placed beneath a patient's skin or muscle and gradually expanded over a period of time to achieve a desired expanded tissue pocket. Soft tissue expansion may have advantages over conventional skin grafting for forming the tissue pocket, including providing a natural appearance in skin texture and color by matching the surrounding skin and an existing blood supply.

Tissue expanders may be used for the surgical reconstruction of the breast following mastectomy for breast cancer. Breast reconstruction is conventionally a two staged process in which the affected breast tissue and skin is removed in a first surgery (mastectomy). A collapsed or partially inflated tissue expander may be placed under the pectoralis major muscle behind the area of the removed breast during the first surgery to create a new breast pocket. The tissue expander may be post-operatively filled with a filler material such as saline or air over a period of time until the breast pocket achieves a desired volume to accommodate a final permanent breast implant. In a second surgery, the tissue expander may be removed and replaced with the final permanent breast implant or the breast may be reconstructed with the patient's own tissue with tissue flap surgery.

Current breast tissue expanders comprise a silicone elastomer shell with an injection port for injection of the filler material. The breast tissue expanders may comprise a conventional round full breast shape, a partial breast shape, or a contour shape for greater lower pole volume expansion of the breast pocket. Some breast tissue expanders may further comprise suture tabs for suturing the tissue expander to fascia on the patient's chest wall. The suture tabs may be small and difficult for the surgeon to access during surgery.

Once implanted under the pectoralis major muscle during the first stage surgery, a fibrous capsule of scar tissue forms over the tissue expander as part of a normal physiologic response of the tissue to a foreign object. It is into this encapsulated breast pocket that the final breast implant will be implanted. The thickness of the capsule may range from thin to heavily-thickened. The formation of the fibrous capsule may cause the breast pocket created by the tissue expander to be of inadequate dimensions or to become an undesirable rounded shape incapable of providing a desired teardrop shaped breast pocket. The teardrop shape of the breast pocket may provide a natural looking breast with either a round or an anatomically shaped final permanent breast implant. The fibrous capsule may also cause capsular contracture in which the breast pocket is constricted, causing deformation of the breast pocket, excessive firmness of the breast pocket, and/or pain.

As a result, in the second surgery, surgeons routinely perform an open capsulotomy in which the breast pocket created by the tissue expander is further surgically modified to achieve the desired size and shape to accommodate the final permanent breast implant. The capsulotomy results in additional surgical time, complexity, and cost. Since the capsulotomy necessarily results in additional trauma to the tissue surrounding the breast pocket, the patient may experience additional pain and a longer recovery time. Further, the patient may develop complications such as tissue necrosis, bleeding, and/or irregularities in the chest wall and/or skin.

### BRIEF SUMMARY

Various embodiments provide systems and methods for a temporary extended tissue expander. Systems and methods according to various aspects of the present invention may comprise an extended tissue expander configured to increase and/or customize the size and/or shape of a tissue pocket beyond the size and/or shape provided by a conventional tissue expander. The extended tissue expander may comprise a tissue expander coupled to an extension portion. In one embodiment, the extended tissue expander may preserve a breast pocket with a desired teardrop shape that does not need extensive surgical modification before placement of a final breast implant or a tissue flap. In another embodiment, the extended tissue expander may be modified to the precise dimensions of a final breast implant for the simple exchange of the extended tissue expander for the final breast implant.

### BRIEF DESCRIPTION OF THE DRAWING FIGURES

A more complete understanding of the present invention may be derived by referring to the detailed description when considered in connection with the following illustrative figures. In the following figures, like reference numbers refer to similar elements and steps throughout the figures.

Elements and steps in the figures are illustrated for simplicity and clarity and have not necessarily been rendered according to any particular sequence or scale. For example, steps that may be performed concurrently or in different order are illustrated in the figures to help to improve understanding of embodiments of the present invention.

The figures described are for illustration purposes only and are not intended to limit the scope of the present disclosure in any way. Various aspects of the present invention may be more fully understood from the detailed description and the accompanying drawing figures, wherein:

FIGS. 1A-1B representatively illustrate an anterior view of an extended tissue expander comprising an extension portion that completely covers and extends beyond a posterior surface of a tissue expander;

FIGS. 2A-2B representatively illustrate a side view of the extended tissue expander comprising the extension portion that completely covers and extends beyond the posterior surface of the tissue expander:

FIG. 3 representatively illustrates a posterior view of the extended tissue expander comprising the extension portion that completely covers and extends beyond the posterior surface of the tissue expander;

FIG. 4 representatively illustrates an anterior view of an extended tissue expander comprising an extension portion that partially covers a posterior surface of a tissue expander;

FIG. 5 representatively illustrates a side view of the extended tissue expander comprising the extension portion that partially covers the posterior surface of the tissue expander;

FIG. 6 representatively illustrates a posterior view of the extended tissue expander comprising the extension portion that partially covers the posterior surface of the tissue expander;

FIG. 7 representatively illustrates a side view of an extended tissue expander comprising an extension portion that extends upward from an upper boundary of a tissue expander;

FIG. 8 representatively illustrates a posterior view of an extended tissue expander comprising the extension portion that extends upward from the upper boundary of the tissue expander;

FIG. 9 representatively illustrates an exemplary extended tissue expander disposed within a breast pocket of a patient;

FIGS. 10A-10C representatively illustrate side views of an extended tissue expander disposed within a breast pocket of a patient after mastectomy and subsequently expanded in the breast pocket;

FIGS. 11A-11C representatively illustrate the modification of an extended tissue expander along a guideline to match the dimensions of a form stable, highly cohesive silicon gel breast implant, shown with anterior views;

FIGS. 12A-12C representatively illustrate the modification of the extended tissue expander along the guideline to match the dimensions of the form stable, highly cohesive silicon gel breast implant, shown with side views; and

FIG. 13 representatively illustrates a method of using an extended tissue expander.

### DETAILED DESCRIPTION OF EXEMPLARY EMBODIMENTS

The present invention may be described in terms of functional block components and various processing steps. Such functional blocks may be realized by any number of components configured to perform the specified functions and achieve the various results. For example, methods and systems according to various aspects of the present invention may be practiced in conjunction with any number of systems and methods for tissue reconstruction, and the system described is merely one exemplary application for the invention. Various representative implementations of the present invention may be applied to any type of conventional tissue expander.

The particular implementations shown and described are illustrative of the invention and its best mode and are not intended to otherwise limit the scope of the present invention in any way. For the sake of brevity, conventional manufacturing, connection, preparation, sterilization, and other functional aspects of the system may not be described in detail. Furthermore, the connecting lines shown in the various figures are intended to represent exemplary functional relationships and/or steps between the various elements. Many alternative or additional functional relationships or physical connections may be present in a practical system.

Various aspects of the invention provide methods, apparatus, and systems for making and using an extended tissue expander system in reconstructive surgeries. A detailed description of various embodiments is provided as a specific enabling disclosure that may be generalized to any application of the disclosed systems and methods in accordance with the various described embodiments.

Various representative implementations of the present invention may be applied to any appropriate system for tissue reconstruction. Certain representative implementations may include, for example, providing any suitable system or method for extending a conventional tissue expander to increase and/or customize the size and/or shape of a tissue pocket for accommodating a final implant or a tissue flap. In one embodiment, the extended tissue expander system may comprise a static nonexpandable extension portion coupled to any conventional inflatable tissue expander, such as a tissue expander used for facial reconstruction or breast reconstruction following mastectomy. In one embodiment, the combination of the static extension portion with the conventional tissue expander may maintain established soft tissue expansion methods and techniques, such as for breast tissue expansion, while providing a larger tissue pocket for accommodating the size and/or shape of the final implant and an improved surgical outcome. In one embodiment as applied to breast reconstruction, the extension portion may provide additional height to the resulting tissue pocket beyond an upper boundary of the inflated tissue expander without anterior expansion away from the chest wall, resulting in a teardrop shaped tissue pocket that may form a more natural tight shape to receive the final breast implant. The tissue expander may comprise any suitable tissue expander for expanding soft tissue, such as a breast, eyelid, and/or lip.

In an exemplary embodiment of the present invention, the extended tissue expander may be configured for submuscular implantation in the breast after mastectomy in a female patient to form a breast pocket comprising expanded skin. In some embodiments, the extended tissue expander may be implanted during a first surgery of a two staged breast reconstruction surgery. In one embodiment, the extended tissue expander may be configured for submuscular implantation in the chest of a male for a two staged male breast augmentation.

The tissue expander of the extended tissue expander may be in an empty collapsed state when surgically implanted or it may be at least partially filled with a filler material such as a saline solution or air. The extended tissue expander may be post-operatively filled with the filler material over time until the breast pocket reaches a desired volume. A second surgery of the two staged surgery may occur weeks to months after the first surgery wherein the extended tissue expander may be removed and replaced with a final breast implant or a tissue flap. An implanted tissue expander may not be intended to be a permanent implant by its manufacturer.

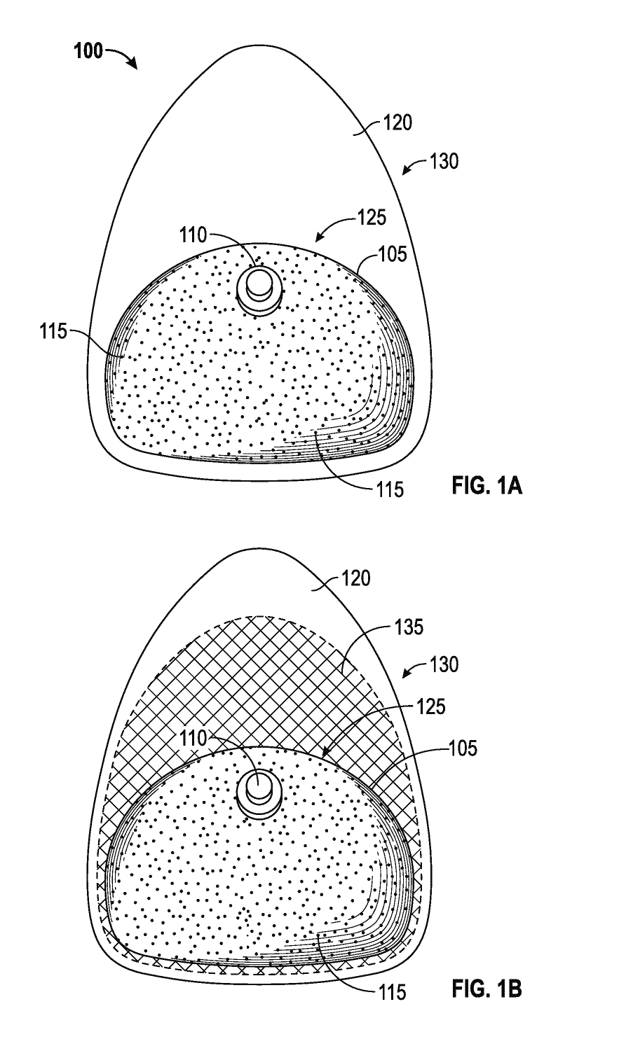

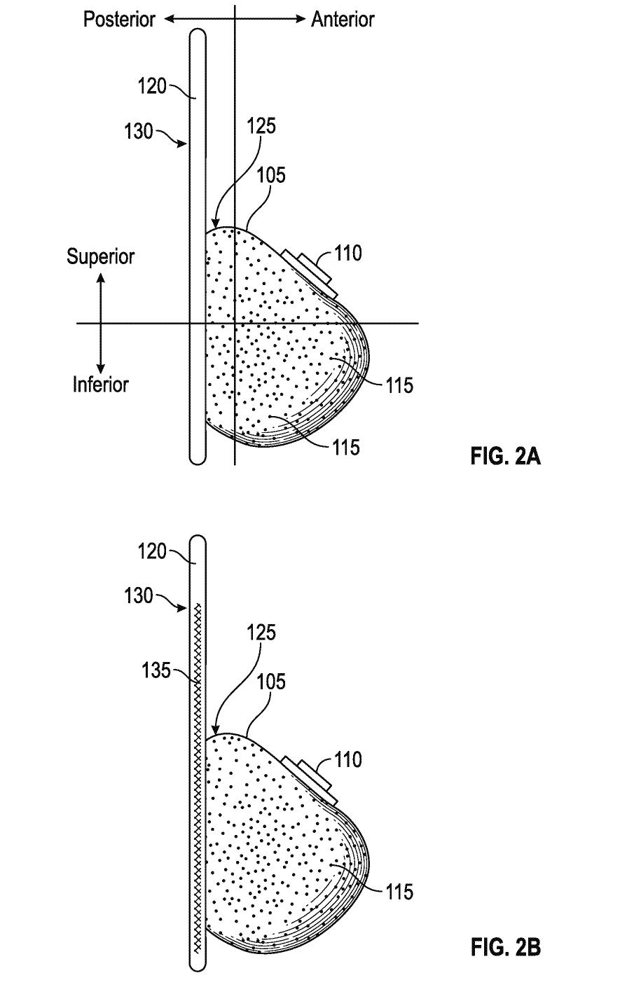

Referring to FIGS. 1A-1B and 2A-2B, exemplary embodiments of the extended tissue expander 100 may comprise an inflatable tissue expander 105 coupled to an extension portion 120. The tissue expander 105 may comprise any suitable system or device for affecting breast tissue and skin expansion or stretching. For example, the tissue expander 105 may comprise an inflatable balloon constructed from silicone elastomer or polypropylene. In some embodiments, the tissue expander 105 may further comprise a self-sealing integral injection port 110, such as a septum, that may be pierced with a hypodermic needle to introduce the filler material into a lumen of the tissue expander 105 for inflation. In other embodiments, the injection port 110 may be located remotely from the tissue expander 105. For example, the injection port 110 may be coupled to the tissue expander 105 through a tubing (not shown) to allow the filler material to flow from the injection port 110 into the lumen of the tissue expander 105.

In some embodiments, the tissue expander 105 may be a contour profile tissue expander for extending a lower portion of the breast pocket. The contour profile tissue expander may comprise or be similar to the Mentors® Contour Profile® Tissue Expander.

In another embodiment, the tissue expander 105 may comprise a shape that provides directional expansion to at least a portion of the breast pocket. For example, the directionally expanding tissue expander 105 may comprise or be similar to Dacron® reinforced smooth tissue expanders produced by Mentor®, such as smooth round, smooth rectangle, smooth crescent, or smooth elliptical tissue expanders. Further, the directionally expanding tissue expander 105 may comprise or be similar to the textured or smooth crescent tissue expanders from Sientra®. Further, the directionally expanding tissue expander 105 may comprise or be similar to the Sientra® ACX™ double chamber anatomical controlled breast tissue expander which may be configured for differential inflation of one or both chambers.

In some embodiments, the tissue expander 105 may be configured to create a full breast shaped breast pocket. For example, the tissue expander 105 may comprise or be similar to any of the Mentor® CPX2™, CPX3™, or CPX™ 4 breast tissue expanders in a low, medium, or tall height. Further, the tissue expander 105 may comprise or be similar to Allergan Natrelle® 133 Tissue Expanders. Further, the tissue expander 105 may comprise or be similar to Sientra® round breast tissue expanders or Sientra® anatomical controlled breast tissue expanders in a low, moderate, or full height.

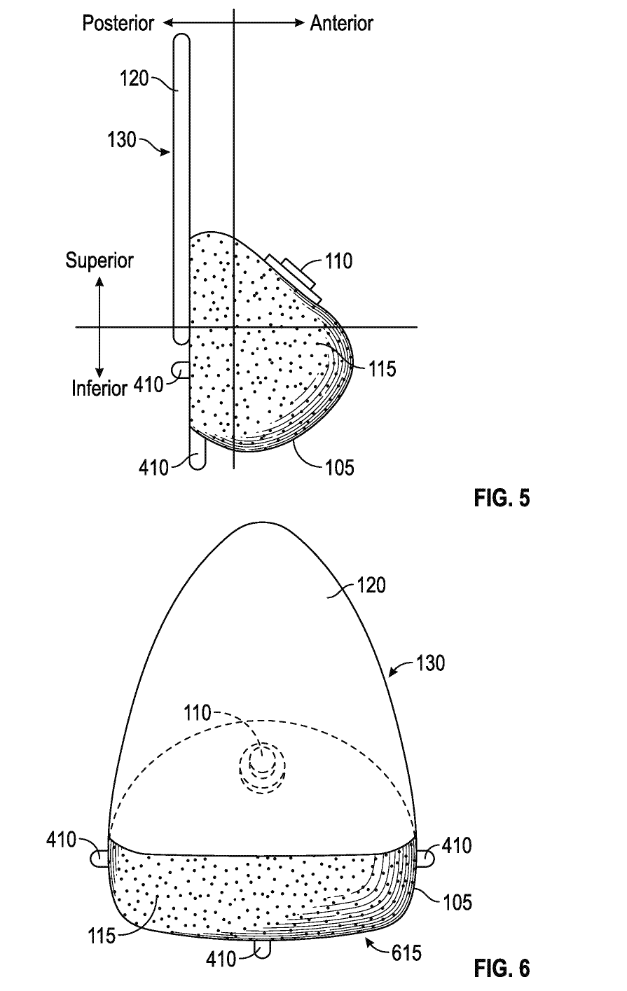

Referring to FIGS. 4-8, in a further embodiment, the tissue expander 105 may comprise one or more suture tabs 410. In one embodiment, the suture tabs 410 may be coupled to the tissue expander 105 and configured to anchor the submuscular position of the tissue expander 105 by allowing the surgeon to suture the suture tabs 410 to fascia, or other native tissues, on the patient's chest wall and/or other underlying tissue to prevent migration and/or rotation of the tissue expander 105. In some embodiments, the suture tabs 410 may comprise an elastomer such as silicone elastomer (sometimes referred to as silicone rubber). In one embodiment, the suture tabs 410 may be reinforced with any biocompatible nonbioabsorbable surgical fabric such as Dacron mesh and/or nylon.

In various embodiments, the suture tabs 410 may be securely coupled to the tissue expander 105 by any suitable method. In one embodiment, the suture tabs 410 may be coupled to the tissue expander 105 with a biocompatible adhesive such as a silicone adhesive. In another embodiment, the suture tabs 410 may be coextruded with the tissue expander 105. In yet another embodiment, the suture tabs 410 may be incorporated into a mold for creating the tissue expander 105.

In various embodiments, the tissue expander 105 may comprise a smooth surface, a surface texture 115, or a combination thereof. The surface texture 115 may promote adherence of the tissue expander 105 to surrounding tissue and/or to the fibrous capsule that forms post-operatively around the extended tissue expander 100. In some embodiments, the surface texture 115 may support immobility of the tissue expander 105 within the breast pocket during expansion of the tissue expander 105. In some embodiments, the surface texture 115 may reduce the incidence of adverse capsular contracture that may cause deformation of the tissue expander 105. In one embodiment, the surface texture 115 of the tissue expander 105 may comprise raised microscale nodules providing a rough feel. For example, the Mentor® tissue expanders described above may comprise SILTEX® surface texturing having microscale nodules. In another embodiment, the surface texture 115 of the tissue expander 105 may comprise surface depressions. For example, the Allergan Natrelle® 133 tissue expanders described above may comprise Biocell® surface texturing having microscale depressions.

The extended tissue expander 100 may further comprise the extension portion 120. The extension portion 120 may comprise any suitable extension element that may be coupled to the tissue expander 105 and provides additional height above the tissue expander 105 within the breast pocket without resulting in substantial anterior expansion away from the chest wall. As a result, in some embodiments, the fibrous capsule may form around both the extension portion 120 and the tissue expander 105 to form a teardrop shaped breast pocket. In one embodiment, the extension portion 120 may have a uniform thickness. In another embodiment, the position of the extension portion 120 may be static in relation to the tissue expander 105. In yet another embodiment, the extension portion 120 may be configured to be sutured to the fascia of the patient's chest wall.

In various embodiments, at least one dimension of the extension portion 120 may be customized during the first surgery of a two staged breast reconstruction to match the desired dimensions of the final encapsulated breast pocket that extends to a desired anatomic boundary of the patient's breast. For example, the extension portion 120 may be trimmed such that the boundaries of the breast pocket may provide a footprint of a breast with the desired dimensions onto the chest wall. In some embodiments, the breast pocket having the footprint of the breast on the chest wall may provide a loose pocket needed for some types of final breast implants such as smooth or textured round breast implants of any size that are configured to move similarly to a natural breast within the loose breast pocket.

In some embodiments, the size and/or shape of the extension portion 120 may be customized during the first surgery to the precise dimensions of a preselected final breast implant, such as a form stable, highly cohesive gel breast implant of any volume. The customized extension portion 120 may provide for the formation of a tight pocket for the simple exchange of the final breast implant for the extended tissue expander 100 and obviating the need for the surgeon to perform a capsulotomy during the second surgery.

Formation of the tight pocket may also prevent the need for capsulorrhaphy in a third surgery to revise the breast pocket to be smaller, resulting in reduced movement and/or altering the position of the final breast implant within the breast pocket. Capsulorrhaphy may be performed to reduce adverse unnatural looking movement or position of the final breast implant such as lateral movement when the patient lies down, migration on the chest wall, and/or rotation of a shaped implant within the breast pocket. Capsulorrhaphy may comprise placing internal sutures along one or more borders of the breast pocket and/or the use of biomaterials such as acellular dermal matrix (ADM's) that provide a cell free lattice of tissue to support an ingrowth of the patient's native tissue, resulting in a smaller breast pocket.

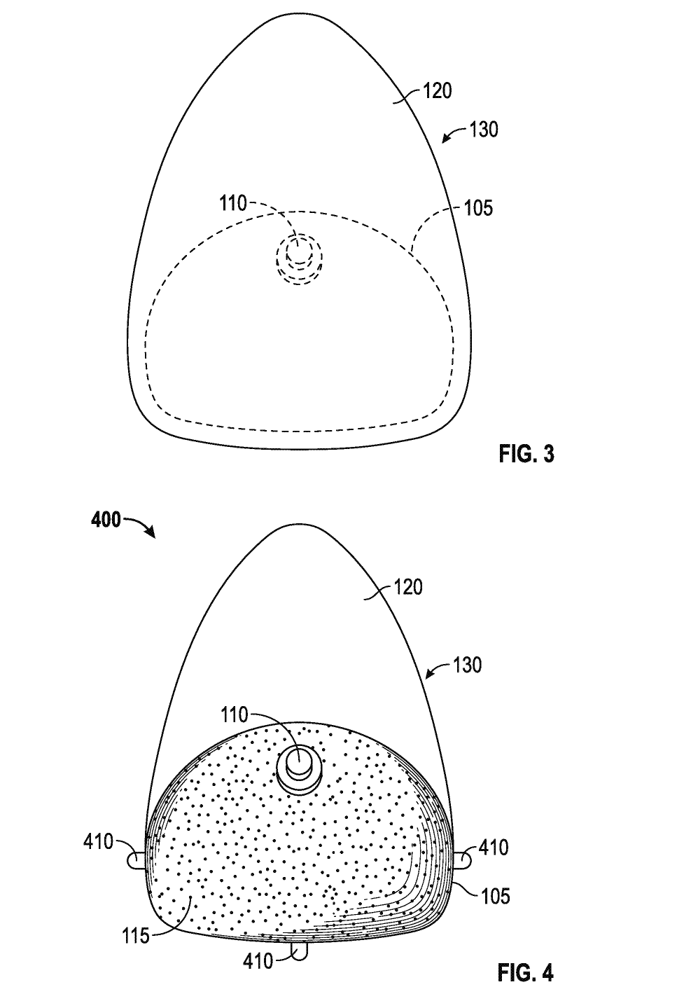

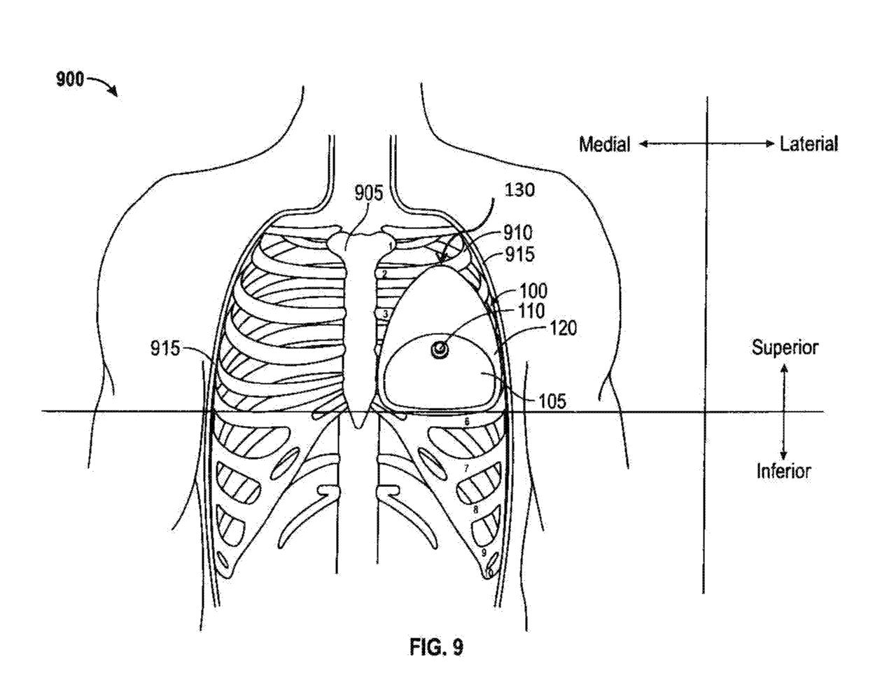

Referring again to FIGS. 1A-1B and 2A-2B, the extended tissue expander 100 may comprise the extension portion 120. In one embodiment, the extension portion 120 may be disposed along a posterior surface of the tissue expander 105 such that the length and width of the extension portion 120 exceeds the length and width of the posterior surface of the tissue expander 105. Further, referring now to FIG. 9, an upper portion 130 of the extension portion 120 may be sufficiently long to extend up to at least about the patient's second rib and/or the upper portion 130 may be at least twice the height of the tissue expander 105. Referring to FIG. 3, a posterior view of the extended tissue expander 100 is illustrated in which the extension portion 120 covers an area greater than the posterior surface of the tissue expander 105. As a result, the tissue expander 105 may not be visible from the posterior view of the extended tissue expander 100.

The extension of the extension portion 120 beyond the dimensions of the posterior surface of the tissue expander 105 may provide the surgeon with ample material for suturing the extended tissue expander 100 to fascia on the patient's chest wall to prevent migration and/or rotation of the extended tissue expander 100 in the breast pocket. In some embodiments, as described below, the extension portion 120 may be configured to be trimmed to the precise dimensions of the final breast implant to provide a tight breast pocket, such as for receiving a form stable, highly cohesive gel breast implant. In some embodiments, the extension portion 120 may be trimmed to provide a footprint of a breast with the desired dimensions onto the chest wall, such as for receiving a smooth or textured round final breast implant.

Referring to FIGS. 4-6, an extension portion 120 may be disposed along the posterior surface of the tissue expander 105 such that the extension portion 120 covers less than the entire posterior surface of the tissue expander 105. As shown in FIG. 6, at least a portion of a posterior surface 615 of the tissue expander 105 may be visible from the posterior view when the tissue expander 105 is coupled to the extension portion 120. In one embodiment, the upper portion 130 of the extension portion 120 may be sufficiently long to extend up to at least about the patient's second rib and/or the upper portion 130 may be at least twice the height of the tissue expander 105.

The upper portion 130 of the extension portion 120 may provide the surgeon with ample material for suturing the extended tissue expander 100 to fascia on the patient's chest wall to prevent migration and/or rotation of the extended tissue expander 100 in the breast pocket. However, in some embodiments, the extension portion 120 may not exceed the width of the tissue expander 105 and/or may not extend below the posterior surface 615 of the tissue expander 105. As a result, the extension portion 120 may not provide lateral and/or inferior material for suturing the extended tissue expander 100 to the fascia. In one embodiment, the suture tabs 410 may be provided on the sides and/or bottom of the tissue expander 105 for additional anchor points for suturing the extended tissue expander 100 to the patient's fascia and/or other native tissues. In some embodiments, the upper portion 130 of this embodiment of the extension portion 120 may be configured to be trimmed to the precise dimensions of the final breast implant to provide a tight breast pocket or to provide the footprint of the breast with the desired dimensions onto the chest wall.

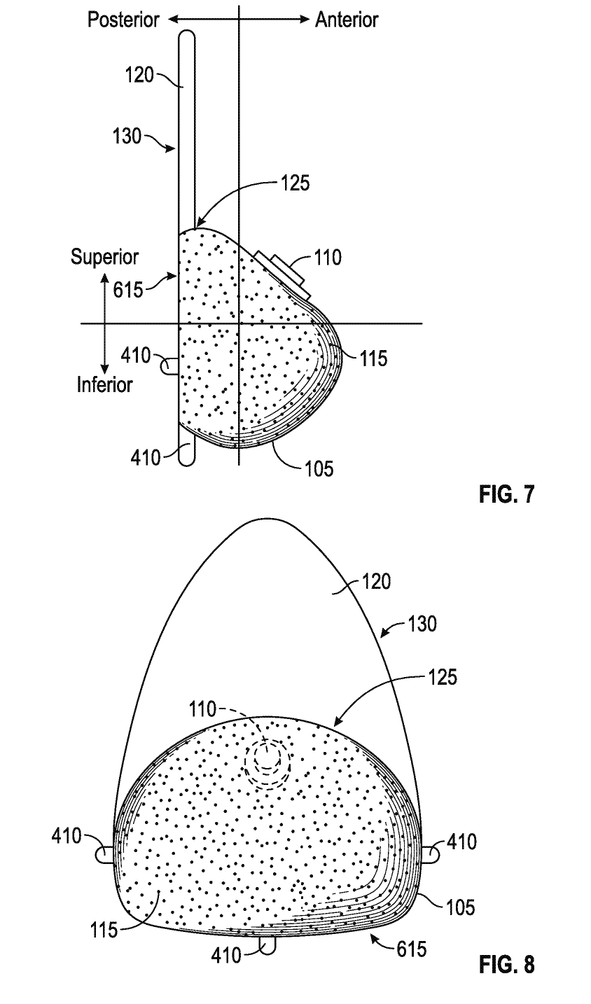

Referring to FIGS. 7 and 8, in another embodiment, an extension portion 120 may be disposed along an upper boundary 125 of the tissue expander 105 and extend upwards from the tissue expander 105. In one embodiment, the entire posterior surface 615 of the tissue expander 105 may be exposed. In one embodiment, the upper portion 130 of the extension portion 120 may be sufficiently long to extend up to at least about the patient's second rib and/or the upper portion 130 may be at least twice the height of the tissue expander 105. The extension portion 120 in this embodiment may only extend from the upper boundary 125 of the tissue expander 105. Therefore, the extension portion 120 may not provide lateral and/or inferior material for suturing the extended tissue expander 100 to the fascia. In one embodiment, the suture tabs 410 may be provided on the sides and/or bottom of the tissue expander 105 for additional anchor points for suturing the extended tissue expander 100 to the fascia.

In various embodiments, the extension portion 120 may comprise any suitable static nonexpandable biocompatible material. The biocompatible material may be a nonbioabsorbable material such that the extension portion 120 remains intact for the duration of the implantation of the extended tissue expander 100. Further, in some embodiments, the extension portion 120 may comprise a substantially resilient material that may be bent or folded for insertion into the breast pocket and subsequently reassume its intended shape. For example, in some embodiments, the extension portion 120 may comprise a sheet of silicone elastomer or polypropylene. In various embodiments, the extension portion 120 may comprise a smooth surface, any appropriate textured surface, or a combination thereof. In some embodiments, the textured surface may comprise the surface texture 115 as described above for the tissue expander 105, such as SILTEX™ or Biocell® surface texturing.

The extension portion 120 may comprise any suitable uniform or variable thickness for maintaining an open space above the tissue expander 105 in the breast pocket by preventing the formation of the fibrous capsule in the space occupied by the extension portion 120. In this manner, the extension portion 120 may provide at least additional height to the resulting breast pocket beyond the upper boundary 125 of the inflated tissue expander 105 without substantial anterior expansion of the breast pocket away from the chest wall, resulting in a teardrop shaped breast pocket that may form a more naturally shaped tight tissue pocket for receiving the final breast implant. For example, in one embodiment, the extension portion 120 may be about 1 to 2 centimeters thick. In another embodiment, the extension portion 120 may be more than 2 centimeters thick. In another embodiment, the extension portion 120 may be less then 1 centimeter thick.

Referring to FIG. 9, the upper portion 130 may be trimmed to extend the breast pocket from the upper boundary 125 of the tissue expander 105 to at least the patient's second rib to maintain an extended open pocket above the tissue expander 105. In one embodiment, the extended tissue expander 100 may be provided with an oversized untrimmed upper portion 130 of the extension portion 120 that may accommodate any patient's height. For example, the height of the upper portion 130 may be longer than the distance between the top of any patient's breast and clavicle. In one embodiment, the untrimmed upper portion 130 may be at least fifteen centimeters.

In another embodiment, a variety of extended tissue expanders 100 may be constructed wherein the untrimmed upper portion 130 may be configured to accommodate the height of a petite patient or a tall patient. For example, the untrimmed upper portion 130 may be 10 centimeters for a petite patient or 15 centimeters for a tall patient. In another embodiment, the untrimmed upper portion 130 may be at least twice the height of the tissue expander 105. In yet another embodiment, the untrimmed upper portion 130 may extend just above the upper boundary 125 of the tissue expander 105 to at least three or more times the height of the tissue expander 105.

Referring to FIG. 1B, an exemplary extension portion 120 may further comprise a surgical fabric 135 that may be imbedded in an inner portion or attached to the extension portion 120. The surgical fabric 135 may comprise any suitable biocompatible, nonbioabsorbable, non-immunogenic, and/or inert surgical fabric configured to provide reinforcement and/or improved mechanical integrity to the silicon elastomer of the extension portion 120. In one embodiment, the surgical fabric 135 may prevent ripping or damage to the extension portion 120 upon suturing the extension portion 120 to fascia on the patient's chest wall and/or as the extended tissue expander 100 moves within the breast pocket. In another embodiment, the surgical fabric 135 may comprise any natural or synthetic material that is biologically compatible with soft tissue and insoluble in body fluids. For example, the surgical fabric 135 may comprise a nonbioabsorbable polymer such as knitted or woven Dacron® mesh (polyethylene terephthalate). In some embodiments, the surgical fabric 135 may be fabricated from polymers such as polyethylene, polypropylene, nylon, polytetrafluorothylene, and/or polyvinylidene fluoride.

The surgical fabric 135 may be embedded in or attached to the extension portion 120 by any suitable method. For example, in one embodiment, the surgical fabric 135 may be attached to an exterior surface of the extension portion 120 (not shown) with a silicone-based adhesive. In another embodiment, the extension portion 120 may be coextruded with the surgical fabric 135. In yet another embodiment, the extension portion 120 may be molded around the surgical fabric 135. In yet another embodiment, the surgical fabric 135 may be embedded in the extension portion 120 by using a sandwich construction method in which the surgical fabric 135 is disposed within the films of the silicone elastomer of the extension portion 120.

Referring to FIGS. 1B and 2B, the surgical fabric 135 may occupy a substantially central portion of the extension portion 120. For example, in some embodiments, the surgical fabric 135 may not extend to the edges of the extension portion 120 to accommodate a need to trim the extension portion 120 to a desired size and/or shape without exposing the raw edges of the surgical fabric 135 upon trimming. As a result, the edges of the surgical fabric 135 may remain sealed within the extension portion 120 after trimming and may still maintain its reinforcement capability for suturing.

In various embodiments, the extension portion 120 may be coupled to the tissue expander 105 by any suitable method. In one embodiment, the extension portion 120 may be secured in a fixed position relative to the tissue expander 105 such that the extension portion 120 cannot rotate or otherwise move in relation to the tissue expander 105. For example, in one embodiment, the extension portion 120 may be coupled directly to the tissue expander 105 with an adhesive such as a silicone adhesive. In another embodiment, the extension portion 120 may be coupled indirectly to the tissue expander 105. For example, the tissue expander 105 may comprise a fastener such as a press stud for attachment of the extension portion 120. In yet another embodiment, the extension portion 120 may be coextruded or included as part of a mold for the tissue expander 105. In this embodiment, the extension portion 120 and the tissue expander 105 may comprise a single continuous device.

In various embodiments, the size and/or shape of the breast pocket may be customized by trimming the extension portion 120 according to an individual patient's anatomy and/or the size of any final breast implant. In one embodiment, the extension portion 120 may be trimmed to the desired boundaries of the final encapsulated breast pocket. For example, the desired boundaries of the final encapsulated breast pocket may be considered to be a footprint of the breast or the desired breast on the chest wall. By customizing the size of the extension portion 120, a substantially simple exchange of the final breast implant for the implanted extended tissue expander 100 may be facilitated, such as during the second stage surgery of breast reconstruction. In some embodiments, the extension portion 120 may be configured to be trimmed, such as with surgical scissors or a scalpel, to substantially match the size and shape of the final breast implant and to fit the submuscular implantation area created by the surgeon in the first stage surgery. For example, referring again to FIG. 9, the outer borders of the extension portion 120 may be trimmed to reach a patient's sternal border 905 medially, lateral chest wall 915 laterally, and second rib 910 superiorly. The inferior border of the extension portion may vary between individual patients according to their natural or desired breast position.

The extension portion 120 of the extended tissue expander 100 may be trimmed to create a breast pocket that may accommodate any suitable final breast implant, such as silicone elastomer or polypropylene breast implants. For example, in one embodiment, the breast implant may comprise any suitable shape such as round, oval, or an anatomic shape such as a teardrop. The breast implant may also contain any suitable filler material such as saline, cohesive silicone gel, and/or a highly cohesive silicone gel. The breast implant may further comprise any suitable surface texture such as smooth or textured.

In various embodiments, the extension portion 120 may be trimmed to substantially exact dimensions of some final breast implants that may require a precisely formed tight breast pocket to maintain the orientation of the final breast implant. Specifically, in one embodiment, the final breast implant may be a form stable, highly cohesive gel breast implant. The form stable, highly cohesive gel breast implant may be filled with a highly cohesive gel that retains the gel within the implant despite an implant rupture. The form stable, highly cohesive gel breast implant provides a tapered anatomical teardrop shape unlike conventional round breast implants. Current examples of form stable, highly cohesive gel breast implants include a Mentor® MemoryShape™ Breast Implant, Allergan Natrelle® Style 410 Breast Implant, and Sientra® Silimed® Breast Implants.

The form stable, highly cohesive gel breast implant may retain its tapered shape regardless of whether it moves or rotates within the breast pocket. As a result, the breast pocket created in the first surgery of a two stage breast reconstruction must also be a teardrop shape that precisely matches the dimensions of the form stable, highly cohesive gel breast implant. If the breast pocket is round or too loose, the form stable, highly cohesive gel breast implant may not maintain a proper position within the breast pocket and the proper breast shape will noticeably be lost.

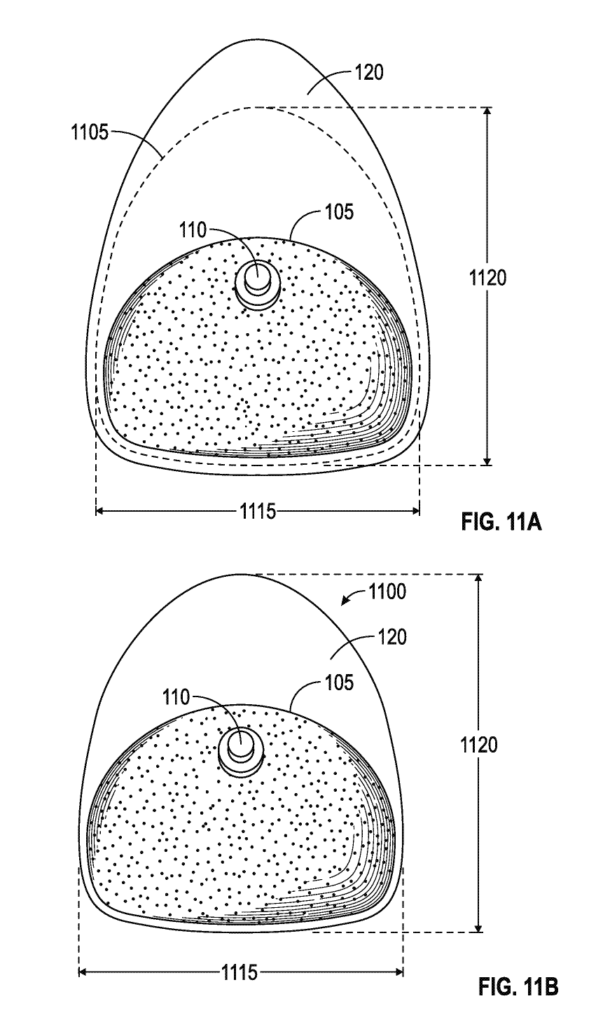

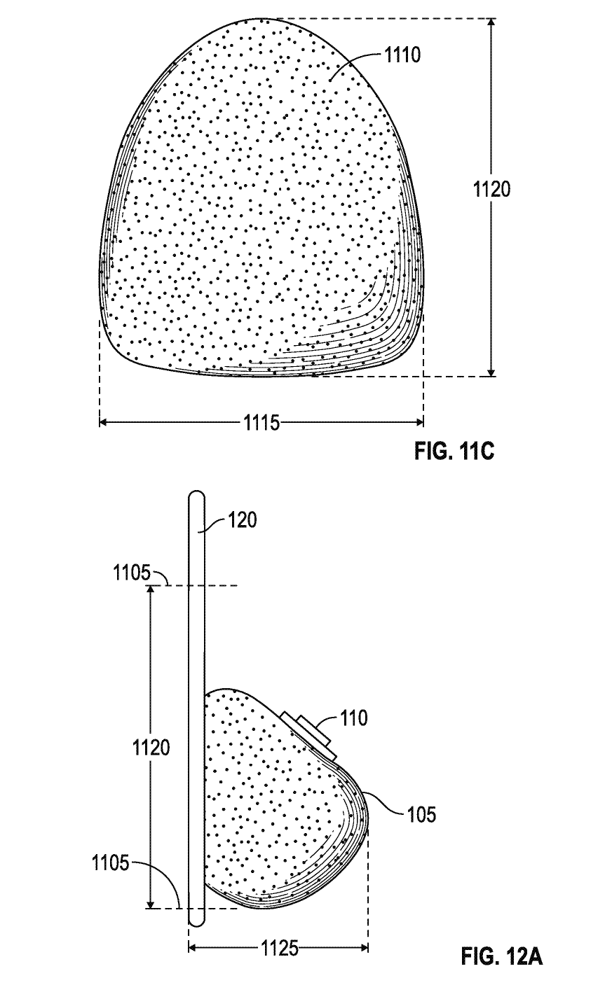

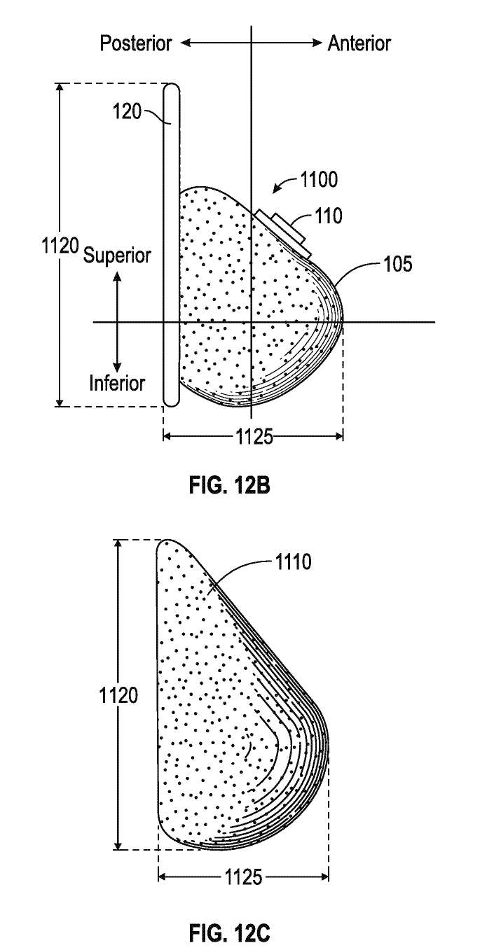

Referring to FIG. 11A-C, the extension portion 120 of the extended tissue expander 100 may be trimmed to substantially exactly match the borders of a form stable, highly cohesive gel breast implant 1110. As shown in FIG. 11A, the surgeon may cut the extension portion 120 along guideline 1105 which matches the height 1120 and width 1115 of the form stable, highly cohesive gel breast implant 1110. FIG. 11B illustrates the resulting trimmed extension portion 120 (1100) that has been cut down to the height 1120 and width 1115 of the highly cohesive gel breast implant 1110. FIGS. 12A-C corresponds to the side views of each anterior view shown in FIGS. 11A-C, respectively. FIGS. 12A-C illustrate that the trimmed extension portion 120 (1100) may result in the extended tissue expander 100 having the substantially precise dimensions of the height 1120, width 1125, and the depth 1125 of the form stable, highly cohesive gel breast implant 1110.

The trimmed extension portion 120 (1100) may be implanted submuscularly into the patient during the first surgery of the two staged breast reconstruction. After the tissue expander 105 has been inflated over time to create the appropriately expanded breast pocket, the trimmed extension portion 120 (1100) may be removed from the breast pocket and exchanged for the highly cohesive gel breast implant 1110 during the second surgery. The breast pocket created by the trimmed extension portion 120 (1100) may reduce or prevent the need for further modification of the breast pocket during the second surgery for the implantation of the form stable, highly cohesive gel breast implant 1110 into the tight breast pocket.

In various embodiments, the guideline 1105 may comprise a virtual line, an imprinted line, a crease, and/or a perforation. In one embodiment, the surgeon may simply measure the dimensions of the final implant and trim the extension portion 120 accordingly along a virtual guideline 1105. In another embodiment, the guideline 1105 may comprise an imprinted line embedded in the extension portion 120 that may correspond to the dimensions of one or more preselected form stable, highly cohesive gel breast implants 1110. In yet another embodiment, the guideline 1105 may comprise a crease or perforation that the surgeon can quickly and easily sever during surgery such as by hand or with surgical scissors. In yet another embodiment, the extension portion 120 may be pre-trimmed to the dimensions of a preselected final breast implant, such as the form stable, highly cohesive gel breast implant 1110, obviating the need for any trimming of the extension portion 120 by the surgeon.

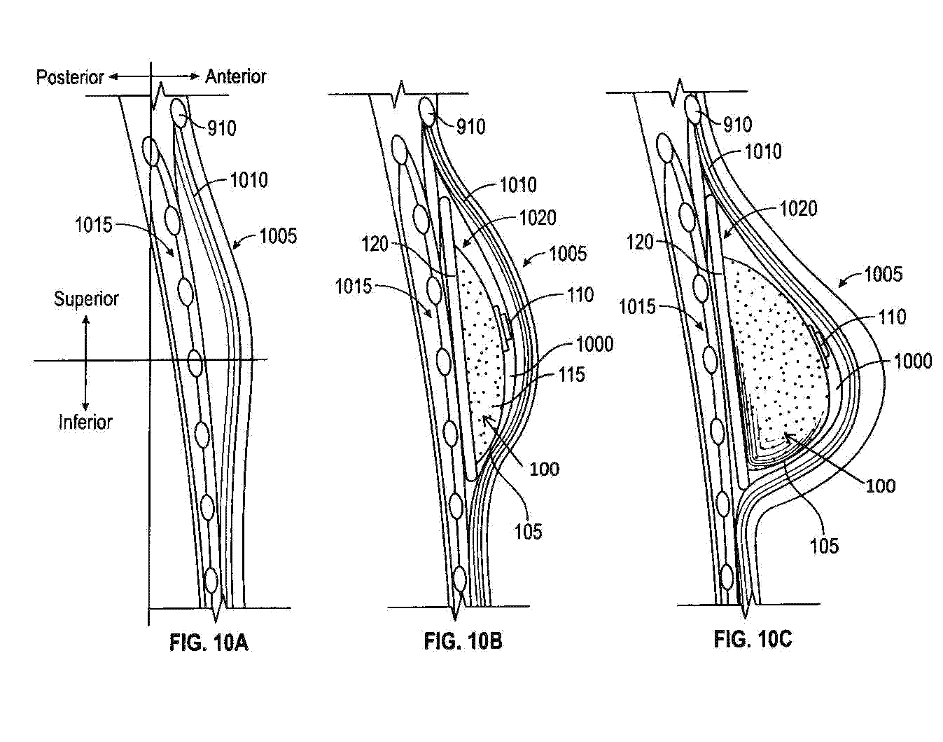

FIGS. 10A-C illustrate the formation of a breast pocket 1000 created in an exemplary first surgery of the two staged breast reconstruction. FIG. 10A illustrates a breast area 1005 wherein cancer affected breast tissue and skin has been removed by mastectomy. FIG. 10B illustrates the implantation of an exemplary extended tissue expander 100 under the pectoralis major muscle 1010 and into the breast pocket 1000 created by the surgeon. In one embodiment, the tissue expander 105 may be at least partially collapsed upon implantation during the first surgery. Accordingly, the breast pocket 1000 is not yet formed to the desired volume needed for the final breast implant.

In various embodiments, the extended tissue expander 100 may be disposed into the breast pocket 1000, wherein the breast pocket 1000 is located in any suitable anatomic soft tissue plane in the breast area 1005. In one embodiment, as shown in FIG. 10A-C, the extended tissue expander 100 may be implanted under the pectoralis major muscle 1010. In another embodiment, the extended tissue expander 100 may be implanted above the pectoralis major muscle 1010 in the subcutaneous plane (not shown). In yet another embodiment, the extended tissue expander 100 may be implanted under other muscles in the chest wall 1015, such as the pectoralis minor, serratus anterior, or the latissimus dorsi (not shown).

In some embodiments, the extended tissue expander 100 may be used to expand the breast pocket in preparation for the second surgery in which the patient receives a tissue flap for breast reconstruction instead of the final breast implant (not shown). In a tissue flap surgery, the patient's own skin, fat, and/or muscle from another part of their body may be removed for use in reconstructing the breast or gradually moved to the breast area without severing the flap's blood supply. In one embodiment, the tissue flap may comprise skin and tissue taken from another part of the patient's body for use in reconstruction of the breast, such as a deep inferior epigastric artery perforator (DIEP) flap, a superior gluteal artery perforator (SGAP) flap, or an inferior gluteal artery perforator (IGAP) flap. In another embodiment, the tissue flap may comprise muscle in addition to skin and tissue taken from another part of the patient's body, such as a transverse rectus abdominus muscle (TRAM) flap.

FIG. 10C illustrates the inflated tissue expander 105 wherein the breast pocket 1000 has expanded to the desired dimensions to accommodate the final breast implant. The patient may then be ready for the second surgery to exchange the extended tissue expander 100 for the final breast implant. In FIGS. 10B and 10C, the extension portion 120 is shown to extend an upper margin 1020 of the breast pocket 1000 (corresponding to the height of the extension portion 120, such as height 1120) without a substantial amount of anterior expansion away from the chest wall 1015. FIG. 10C illustrates the breast pocket 1000 having the teardrop shape created by the extended tissue expander 100.

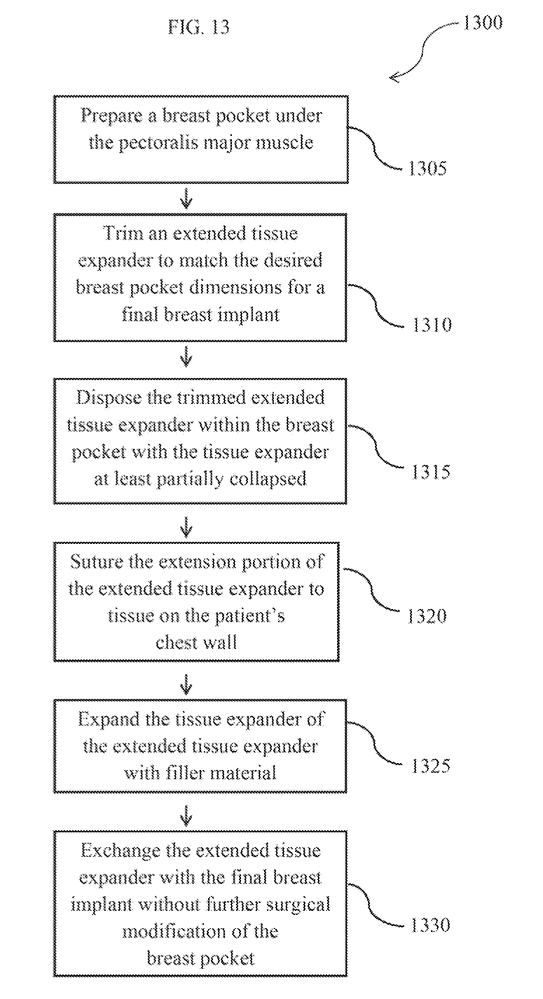

FIG. 13 illustrates an exemplary method of preserving an upper margin 1020 of the breast pocket 1000 in the first surgery of a two staged breast reconstruction (1300). A method of using the extended tissue expander 100, according to various aspects of the present invention, may comprise surgically preparing a breast pocket 1000 under the pectoralis major muscle 1010 or other suitable anatomic soft tissue plane in the breast area 1005 for implantation of the extended tissue expander 100 in the first surgery (1305). In one embodiment, the extended tissue expander 100 may comprise an extension portion 120 extending upwards past an upper boundary 125 of the tissue expander 105. In a further embodiment, the extension portion 120 may be configured to preserve the upper margin 1020 of the breast pocket 1000 to form a teardrop shaped breast pocket 1000 until the second surgery of the two staged breast reconstruction. The method of using the extended tissue expander 100 may further comprise trimming the outer borders of the extended tissue expander 100, such as along guideline 1105, to substantially match the desired breast pocket dimensions for the final breast implant, such as wherein the final breast implant is a form stable, highly cohesive gel breast implant (1310). In one embodiment, the outer borders of the extended tissue expander 100 may be trimmed to substantially match the desired boundaries of the final encapsulated breast pocket for use with a smooth or textured round final breast implant. The trimmed extended tissue expander 100 may be disposed within the breast pocket 1000 with the tissue expander 105 at least partially collapsed (1315). The trimmed extension portion 120 may be sutured to the fascia on the patient's chest wall to secure and stabilize the position of the extended tissue expander 100 in the breast pocket 1000 (1320). In one embodiment, the tissue expander 105 may be filled with filler material during and/or after surgery to gradually expand the tissue expander 105 over time until the breast pocket 1000 reaches a desired volume to accommodate the final breast implant (1325). The extended tissue expander 100 may then be exchanged with the final breast implant without further surgical modification of the breast pocket 1000 (1330).

In the foregoing description, the invention has been described with reference to specific exemplary embodiments. Various modifications and changes may be made, however, without departing from the scope of the present invention as set forth. The description and figures are to be regarded in an illustrative manner, rather than a restrictive one and all such modifications are intended to be included within the scope of the present invention. Accordingly, the scope of the invention should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present invention and are accordingly not limited to the specific configuration recited in the specific examples.

Benefits, other advantages and solutions to problems have been described above with regard to particular embodiments. Any benefit, advantage, solution to problems or any element that may cause any particular benefit, advantage or solution to occur or to become more pronounced, however, is not to be construed as a critical, required or essential feature or component.

The terms “comprises”, “comprising”, or any variation thereof, are intended to reference a non-exclusive inclusion, such that a process, method, article, composition, system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

The present invention has been described above with reference to an exemplary embodiment. However, changes and modifications may be made to the exemplary embodiment without departing from the scope of the present invention. These and other changes or modifications are intended to be included within the scope of the present invention.

1-30. (canceled)

31. An extended tissue expander for maintaining a breast pocket in a patient during the formation of a final encapsulated breast pocket that is configured to receive a pre-selected final breast implant or tissue flap, the extended tissue expander comprising:
a breast tissue expander; and
an extension portion coupled to the breast tissue expander, wherein the extension portion:
extends superiorly past an upper boundary of the breast tissue expander, and
is capable of maintaining an extended open pocket superiorly to the breast tissue expander to substantially correlate the dimensions of the extended tissue expander to at least one of:
the desired dimensions of the final encapsulated breast pocket that extends to an anatomic boundary of the patient's breast area; and
the dimensions of the pre-selected final breast implant to provide a tight final encapsulated breast pocket for the pre-selected final breast implant.

32. The extended tissue expander of claim 31, wherein the pre-selected final breast implant is a form stable, highly-cohesive silicone gel breast implant.

33. The extended tissue expander of claim 31, wherein the extension portion has a substantially uniform thickness.

34. The extended tissue expander of claim 31, wherein the extension portion at least partially covers a posterior surface of the breast tissue expander when the breast tissue expander is at least partially inflated.

35. The extended tissue expander of claim 31, wherein the extension portion further extends from at least a medial border to at least a lateral border of the breast tissue expander.

36. The extended tissue expander of claim 35, wherein the extension portion further extends beyond the medial border and the lateral border of the breast tissue expander when the breast tissue expander is in a deflated state.

37. The extended tissue expander of claim 36, wherein the extension portion further extends to at least a sternal border medially, a lateral chest wall laterally, and a clavicle superiorly when the extended tissue expander is implanted into the patient.

38. The extended tissue expander of claim 36, wherein the extension portion further extends below an inferior border of the breast tissue expander when the breast tissue expander is in a deflated state.

39. The extended tissue expander of claim 31, wherein the extension portion is adapted to be modified by mechanical trimming.

40. The extended tissue expander of claim 31, wherein the extension portion has a minimal thickness configured to maintain the breast pocket without causing unnecessary anterior expansion away from the patient's chest wall.

41. The extended tissue expander of claim 31, wherein the extension portion comprises a suturable material configured to be sutured to tissue on the patient's chest wall.

42. The extended tissue expander of claim 41, wherein the suturable material comprises an embedded surgical fabric.

43. The extended tissue expander of claim 31, wherein the extension portion comprises a shape configured to provide a teardrop shaped final encapsulated breast pocket when the breast tissue expander is expanded to a desired volume.

44. The extended tissue expander of claim 31, wherein the extension portion is permanently affixed to the breast tissue expander.

45. An extended tissue expander for maintaining a breast pocket in a patient during the formation of a final encapsulated breast pocket that is configured to receive a pre-selected final breast implant or tissue flap, the extended tissue expander comprising:
a breast tissue expander; and
an extension portion coupled to the breast tissue expander, wherein the extension portion:
extends superiorly past an upper boundary of the breast tissue expander and extends from at least a medial border to a lateral border of the breast tissue expander, and
is capable of maintaining an extended open pocket superiorly to the breast tissue expander to substantially correlate the dimensions of the extended tissue expander to at least one of:
the desired dimensions of the final encapsulated breast pocket that extends to an anatomic boundary of the patient's breast area; and
the dimensions of the pre-selected final breast implant to provide a tight breast pocket for the pre-selected final breast implant.

46. The extended tissue expander of claim 45, wherein the extension portion further extends beyond at least one of a medial, lateral, and inferior boundary of the breast tissue expander when the breast tissue expander is in a deflated state.

47. The extended tissue expander of claim 45, wherein the extension portion is adapted to be modified by mechanical trimming.

48. The extended tissue expander of claim 45, wherein the extension portion comprises a suturable material configured to be sutured to tissue on the patient's chest wall.

49. The extended tissue expander of claim 45, wherein the extension portion has a minimal thickness configured to maintain the breast pocket without causing unnecessary anterior expansion away from the patient's chest wall.

50. The extended tissue expander of claim 45, wherein the extension portion comprises a shape configured to provide a teardrop shaped final encapsulated breast pocket when the breast tissue expander is expanded to a desired volume.

51. The extended tissue expander of claim 45, wherein the pre-selected final breast implant is a form stable, highly-cohesive silicone gel breast implant.

52. The extended tissue expander of claim 45, wherein the extension portion has a substantially uniform thickness.

53. An extended tissue expander for maintaining a breast pocket in a patient during the formation of a final encapsulated breast pocket that is configured to receive a pre-selected final breast implant or tissue flap, the extended tissue expander comprising:
a breast tissue expander; and
an extension portion permanently fixed to the breast tissue expander, wherein the extension portion:
extends superiorly past an upper boundary of the breast tissue expander, and
extends past at least one of a medial, lateral, and inferior boundary of the breast tissue expander,
wherein the extension portion comprises:
a suturable material capable of being sutured to tissue on the patient's chest wall, and
a mechanically trimmable material capable of maintaining an extended open pocket superiorly to the breast tissue expander to substantially correlate the dimensions of the extended tissue expander to at least one of:
the desired dimensions of the final encapsulated breast pocket that extends to an anatomic boundary of the patient's breast area; and
the dimensions of the pre-selected final breast implant to provide a tight final encapsulated breast pocket for the pre-selected final breast implant.

54. A method of maintaining a breast pocket in a patient during the formation of a final encapsulated breast pocket that is configured to receive a pre-selected final breast implant or tissue flap after mastectomy for breast reconstruction, the method comprising:
preparing a breast pocket for implantation of an extended tissue expander in a first surgery of a multi-staged breast reconstruction, wherein the extended tissue expander comprises:
a breast tissue expander; and
an extension portion coupled to the breast tissue expander that extends superiorly past an upper boundary of the breast tissue expander and is capable of maintaining an extended open pocket superiorly to the breast tissue expander to substantially correlate the dimensions of the extended tissue expander to at least one of:
the desired dimensions of the final encapsulated breast pocket that extends to an anatomic boundary of the patient's breast area; and
the dimensions of the pre-selected final breast implant to provide a tight final encapsulated breast pocket for the pre-selected final breast implant, and
wherein the extension portion is configured to maintain the open pocket until a subsequent surgery of the multi-staged breast reconstruction;
trimming at least one dimension of the extension portion to substantially correlate the dimensions of the extended tissue expander to at least one of the desired dimensions of the final encapsulated breast pocket and the dimensions of the pre-selected final breast implant; and
disposing the trimmed extended tissue expander into the breast pocket, wherein the breast tissue expander is at least partially collapsed.

55. The method of claim 54, wherein the desired dimensions of the final encapsulated breast pocket provide a loose final encapsulated breast pocket adapted to receive at least one of a smooth and textured round breast implant by extending to a desired anatomic boundary of the patient's breast area.

56. The method of claim 54, wherein the trimming the extension portion to substantially correlate the dimensions of the extended tissue expander to the dimensions of the pre-selected final breast implant provides the tight final encapsulated breast pocket adapted to receive a form stable, highly cohesive gel breast implant.

57. The method of claim 54, further comprising measuring at least one of:
a footprint of the breast on the patient's chest wall correlating to the desired dimensions of the final encapsulated breast pocket; and
the dimensions of the pre-selected final breast implant.

58. The method of claim 54, further comprising removing the extended tissue expander from the final encapsulated breast pocket in the subsequent surgery of the multi-staged breast reconstruction and disposing the pre-selected final breast implant into the breast pocket without surgical adjustment of the margins of the final encapsulated breast pocket.

59. The method of claim 54, further comprising expanding the extended tissue expander with a filler material.

60. The method of claim 59, wherein the final encapsulated breast pocket formed from the expansion of the extended tissue expander comprises a teardrop shape.
